(19) United States
(12) Patent Application Publication
(10) Pub. No.: US 2010/0168568 A1
(43) Pub. Date: Jul. 1, 2010

(54) Combined Diagnostic and Therapeutic Device Using Aligned Energy Beams

(75) Inventor: John W. Sliwa, Los Altos Hills, CA (US)

Correspondence Address: SJM/AFD - DYKEMA;c/o CPA Global, P.O. Box 52050, Minneapolis, MN, 55402, US

(73) Assignee: St. Jude Medical, Atrial Fibrillation Division Inc., Minnetonka, MN (US)

(21) Appl. No.: 12/346,042

(22) Filed: Dec. 30, 2008

(51) Int. Cl. A61B 18/14; A61B 8/00; A61N 1/30

(52) U.S. Cl. 600/439; 606/41; 604/20

(57) ABSTRACT

A device for both providing therapy to a tissue and detecting a characteristic of said tissue is provided. The device includes a deformable tubular body such as a catheter or scope. An electrode is supported by the body and configured to deliver therapeutic energy to the tissue along a first path. The electrode may, for example, be used in cardiac ablation and the therapeutic energy may comprise any common ablation energy modality including radio waves or ultrasound waves. The device further includes an acoustic transducer supported by the body and configured to receive acoustic energy along a second path. The transducer may also transmit acoustic energy. The first and second paths are aligned and may be parallel or overlap, for example. The physical relationship of the electrode and transducer and the alignment of the energy paths eliminates the need to register the spatial coordinates of the therapeutic and diagnostic elements.

### BACKGROUND OF THE INVENTION

a. Field of the Invention

This invention relates to devices for the diagnosis and treatment of tissue in a body. In particular, the instant invention relates to a device using aligned energy beams for both diagnosis and treatment of the tissue to thereby eliminate the need to register the spatial coordinates of separate diagnostic and treatment devices.

b. Background Art

It is common to diagnose or assess the state of tissue in a body contemporaneously with treatment of the tissue by either imaging the affected tissue or sensing one or more characteristics of the tissue. Cardiac tissue undergoing ablation to create tissue necrosis, for example, is often imaged using a separate internal or external imaging device (e.g., an intravascular ultrasound (IVUS) catheter). Ablation of cardiac tissue is used to correct conditions such as atrial arrhythmia (including, but not limited to, ectopic atrial tachycardia, atrial fibrillation, and atrial flutter). Arrhythmia can create a variety of dangerous conditions including irregular heart rates, loss of synchronous atrioventricular contractions and stasis of blood flow which can lead to a variety of ailments and even death. It is believed that the primary cause of atrial arrhythmia is stray electrical signals within the left or right atrium of the heart. An ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias. An imaging device or other diagnostic device is used contemporaneously with the ablation catheter to assess the effectiveness of the procedure by, for example, insuring proper placement of the lesion.

Because different devices are commonly used for diagnosis and treatment of tissue during cardiac ablation and other procedures, it is necessary to register the spatial coordinates of the devices within a common coordinate system to guard against intended or unintended relative motion of the devices. Absent registration of the two devices, it would not be possible to determine, for example, whether the tissue that is being assessed is the same tissue that is being treated. Conventional methods for registration of diagnostic and treatment devices, however, have several significant drawbacks. Most registration methods require that additional components (e.g., positions sensors or fiducial markers) be affixed to the diagnostic and treatment devices—devices in which there are already significant size constraints and in which biological interaction is a significant concern. Registration also generally requires significant computational resources thereby reducing resources available to other components of diagnostic and/or treatment systems.

The inventor herein has recognized a need for a device for both providing therapy to a tissue and detecting a characteristic of the tissue that will minimize and/or eliminate one or more of the above-identified deficiencies.

### BRIEF SUMMARY OF THE INVENTION

It is desirable to provide a device for both providing therapy to a tissue and detecting a characteristic of the tissue. In particular, it is desirable to provide a device that eliminates the need to register separate diagnostic and treatment devices.

A device for both providing therapy to a tissue and detecting a characteristic of said tissue in accordance with one embodiment of the present invention includes a deformable, tubular body. The deformable, tubular body may, for example, comprise a catheter or scope. The device further includes an electrode supported by the body and configured to deliver therapeutic energy to the tissue along a first path. The device further includes an acoustic transducer supported by the body and configured to receive acoustic energy along a second path. The first and second path are aligned and may, for example, be parallel to one another or overlap one another (e.g., along a common central axis). In accordance with another embodiment of the invention, the acoustic transducer may further transmit acoustic energy along a third path with the received acoustic energy comprising a portion of the transmitted acoustic energy reflected by the tissue and with the third path aligned with the first and/or second paths.

A device in accordance with the present invention is advantageous because the physical relationship between the electrode and acoustic transducer and the alignment of the paths traversed by the energy from the electrode and to or from the acoustic transducer, inherently registers the diagnostic and therapeutic components of the device. As a result, conventional methods used for registration of separate diagnostic and therapeutic devices are not required thereby eliminating any need for additional position tracking components (e.g., position sensors or fiducial markers) and reducing the demand on computational resources.

The foregoing and other aspects, features, details, utilities and advantages of the present invention will be apparent from reading the following description and claims, and from reviewing the accompanying drawings.

### BRIEF DESCRIPTION OF THE DRAWINGS

FIG. 1 is diagrammatic and block diagram of a device in accordance with one embodiment of the present teachings.

FIG. 2 is a diagrammatic view of a portion of the device of FIG. 1.

FIG. 3 is a diagrammatic view illustrating a portion of a device in accordance with another embodiment of the present teachings.

FIG. 4 is a diagrammatic view illustrating a portion of a device in accordance with another embodiment of the present teachings.

FIG. 5 is a diagrammatic view illustrating a portion of a device in accordance with another embodiment of the present teachings.

FIG. 6 is a schematic view illustrating a portion of a device in accordance with another embodiment of the present teachings.

### DETAILED DESCRIPTION OF EMBODIMENTS OF THE INVENTION

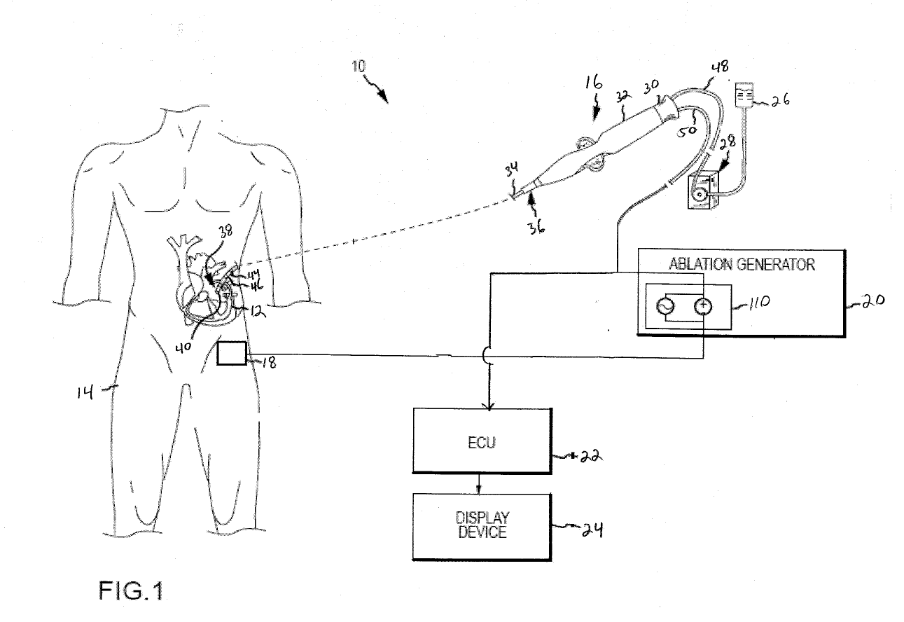

Referring now to the drawings wherein like reference numerals are used to identify identical components in the various views, FIGS. 1 illustrates one embodiment of a device 10 for both providing therapy to a tissue and detecting a characteristic of the tissue 12 in a body 14. In the illustrated embodiment, tissue 12 comprises heart or cardiac tissue. It should be understood, however, that the present invention may be used to provide therapy to, and detect characteristics of, a variety of body tissues. In the illustrated embodiment, device 10 includes an ablation catheter 16, patch electrode 18, an ablation generator 20, an electronic control unit 22 and a display device 24. Although the illustrated embodiment is used for ablation of tissue 12, it should be understood that the invention as described and claimed herein can be configured for other uses. In particular, the present invention can from a part of various endoscopic systems used to provide therapy to, and detect characteristics of, various tissues, for example.

Referring again to FIG. 1, ablation catheter 16 is provided for examination, diagnosis and treatment of internal body tissues such as tissue 12. In accordance with one embodiment of the invention, catheter 16 comprises an irrigated radio-frequency (RF) ablation catheter. It should be understood, however, that the present invention can be implemented and practiced regardless of the type of ablation energy provided (e.g., cryoablation, ultrasound, laser, microwave, electroporation etc.). Catheter 16 is connected to a fluid source 26 having a biocompatible fluid such as saline through a pump 28 (which may comprise, for example, a fixed rate roller pump or variable volume syringe pump with a gravity feed supply from fluid source 26 as shown) for irrigation. Catheter 16 is also electrically connected to ablation generator 20 for delivery of RF energy. Catheter 16 may include a cable connector or interface 30 and a handle 32. In accordance with the present invention, catheter 16 further includes a deformable, tubular body or shaft 34 having a proximal end 36 and a distal 38 end (as used herein, “proximal” refers to a direction toward the end of the catheter near the clinician, and “distal” refers to a direction away from the clinician and (generally) inside the body of a patient), means, such as an electrode 40, for delivering therapeutic energy to tissue 12, and means, such as an acoustic transducer 42 (see FIG. 2), for receiving acoustic energy. Catheter 16 may further include electrodes 44, 46 and other conventional components not illustrated herein such as a temperature sensor, additional electrodes, and corresponding conductors or leads.

Connector 30 provides mechanical, fluid and electrical connection(s) for cables 48, 50 extending from pump 28 and ablation generator 20, respectively. Connector 30 is conventional in the art and is disposed at a proximal end of catheter 16.

Handle 32 provides a location for the clinician to hold catheter 16 and may further provides means for steering or guiding shaft 34 within body 14. For example, handle 32 may include means to change the length of a guidewire extending through catheter 16 to distal end 38 of shaft 34 to steer shaft 34. Handle 32 is also conventional in the art and it will be understood that the construction of handle 32 may vary.

Shaft 34 is an elongated, tubular, flexible body configured for movement within body 14. Shaft 34 supports electrodes 40, 44, 46, transducer 42, associated conductors, and possibly additional electronics used for signal processing or conditioning. Shaft 34 may also permit transport, delivery and/or removal of fluids (including irrigation fluids and bodily fluids), medicines, and/or surgical tools or instruments. Shaft 34 may be made from conventional materials such as polyurethane and defines one or more lumens configured to house and/or transport electrical conductors, fluids or surgical tools. Shaft 34 may be introduced into a blood vessel or other structure within body 16 through a conventional introducer. Shaft 34 may then be steered or guided through body 16 to a desired location such as tissue 12 with guide wires or other means known in the art.

Electrode 40 may comprise an ablation tip electrode as shown in the illustrated embodiment and is located at distal end 38 of shaft 34. Electrode 40 is supported by shaft 34 and is configured to deliver therapeutic energy (illustrated by solid lines 52) to tissue 12 along a first path (illustrated by arrowhead 54). In accordance with the illustrated embodiment of the invention, the therapeutic energy 52 is used to ablate tissue 12. Electrode 40 may also be used for other diagnostic and therapeutic purposes including, for example, electrophysiological studies, catheter identification and location, pacing, and cardiac mapping. In the illustrated embodiment, the therapeutic energy 52 comprises electromagnetic radiation emitted from electrode 40 and, in particular, radio waves. It should be understood, however, that electrode 40 could be configured to deliver a variety of therapeutic energy including other forms of electromagnetic radiation such as microwaves, thermal energy such as cryogenic energy, acoustic energy such as high intensity focused ultrasound (HIFU), and optical or laser energy. Electrode 40 could also deliver therapeutic energy in the form of electrical energy sufficient to cause electroporation of cell membranes in tissue 12. Electrode 40 may comprise a disposable element that can be removed from shaft 34 following use to permit reuse of shaft 34.

Transducer 42 is provided to receive acoustic energy (illustrated as dashed lines 56) along a second path (illustrated by arrowhead 58). The acoustic energy 56 received by transducer 42 may be generated by tissue 12 as a result of natural biological processes or in response to the impact of therapeutic energy 52. Alternatively, transducer 42 may also be configured to transmit acoustic energy (illustrated as dashed waves 60) along a third path (illustrated by arrowhead 62) and the received acoustic energy 56 may comprise a portion of the transmitted acoustic energy 60 reflected by tissue 12. Transducer 42 may have a structure such as one of the structures described and illustrated in any of U.S. Published Patent Application No. 20080195003, U.S. Published Patent Application No. 20080194967, U.S. Published Patent Application No. 20080194965 U.S. Published Patent Application No. 20080189932 or U.S. Published Patent Application No. 20060236526, all of which are assigned to St. Jude Medical, Atrial Fibrillation Division, Inc. and the entire disclosures of which are incorporated herein by reference. Transducer 42 may, for example, include a single or multiple transducer elements, may be round or flat and rectilinear, and may comprise a phased array transducer. Transducer 42 may include an acoustic lens 64, a matching layer coupled to the face of transducer 42 and/or a standoff (e.g., a biocompatible gel and/or a saline solution dispensed between the tissue 12 and the electrode either directly or into a fluid membrane disposed adjacent the tissue 12 and the transducer 42) focusing waves generated through a piezoelectric element 66. Piezoelectric element 66 may also serve as a fiducial marker for fluoroscopic imaging of the tissue 12 and surrounding area. Transducer 42 generates a signal indicative of the acoustic energy 56 received by transducer 42 and transmits the signal to ECU 22.

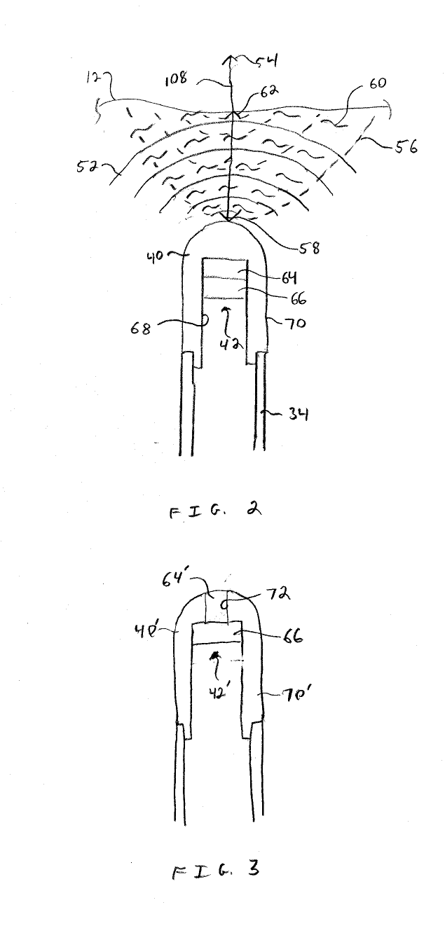

Transducer 42 is supported by shaft 34. As shown in FIG. 2, in one embodiment of the invention, transducer 42 may be further disposed within a chamber 68 defined by the wall 70 or walls of electrode 40. As a result, transducer is nearer proximal end 36 of shaft 34 than the distal tip of electrode 40 and receives and/or transmits acoustic energy 56, 60 through wall 70 of electrode 40. The lateral thickness of wall 70 may be reduced (not shown) in the area through which energy 56, 60 passes to increase through-transmission and receipt of energy 56, 60. Referring to FIG. 3, in an alternative embodiment of the invention, a transducer 42′ receives and/or transmits acoustic energy 56, 60 through an aperture 72, or window, in a wall 70′ of an electrode 40′. The aperture 72 in wall 70′ may be covered by a lens 64′, a matching layer coupled to the face of transducer 42′ and/or a standoff (e.g., a biocompatible gel and/or a saline solution dispensed between the tissue 12 and the electrode either directly or into a fluid membrane disposed adjacent the tissue 12 and the transducer 42′) or another focusing structure for acoustic energy 56, 60. It should be noted that any disturbance or interference with the acoustic energy 56, 60, received or transmitted by transducer 42 or 42′ resulting from the electrode wall 70 or a lens 64′ or other object in the path of energy 56, 60 can be controlled with appropriate design of these elements (e.g., material selection and physical dimensions) and that the elements may even provide impedance matching to actually reduce any interference.

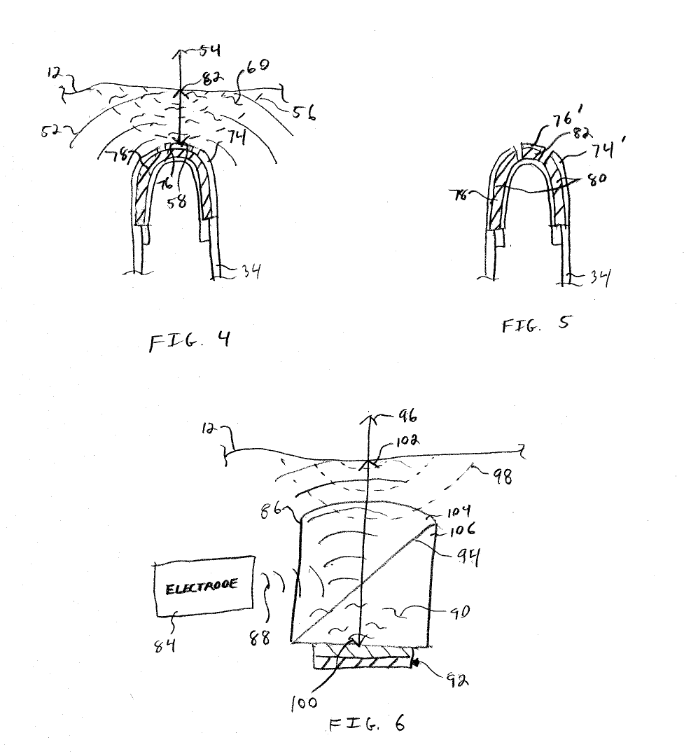

Referring now to FIG. 4, another embodiment of the present invention is illustrated in which an electrode 74 generates therapeutic energy in the form of acoustic energy and, in particular, high intensity focused ultrasound (HIFU). In this embodiment, electrode 74 and acoustic transducer 76 have a common piezoelectric layer 78. As in the case of electrodes 40 and ‘40’, electrode 74 is configured to deliver therapeutic energy (illustrated by solid lines 52) to tissue 12 along a first path (illustrated by arrowhead 54) while transducer 76 is configured to receive acoustic energy (illustrated as dashed lines 56) along a second path (illustrated by arrowhead 58) and may also be configured to transmit acoustic energy (illustrated as dashed waves 60) along a third path (illustrated by arrowhead 62) with the received acoustic energy 56 comprising a portion of the transmitted acoustic energy 60 reflected by tissue 12. Referring to FIG. 5, in another embodiment of the invention, the portion 80 of the piezoelectric layer 78′ forming a part of electrode 74′ is isolated from a portion 82 of the piezoelectric layer 78′ forming transducer 76′ by removing a portion of the piezoelectric layer 78′ on either side of transducer 76′ with, for example, a laser, in order to improve acoustic sensitivity.

Referring now to FIG. 6, another embodiment of the present invention is schematically illustrated in which an electrode 84 generates therapeutic energy in the form of laser energy or another form of optical energy. In this embodiment, a lens 86 or standoff used to direct and focus the optical energy (illustrated by solid lines 88) generated by electrode 84 and any acoustic energy (illustrated by dashed waves 90) generated by a transducer 92. Lens 86 includes a reflective element 94 such as a mirror that alters the path of, and redirects, optical energy 88 along a path (illustrated by arrowhead 96) towards tissue 12. Transducer 92 receives acoustic energy (illustrated as dashed lines 98) along a second path (illustrated by arrowhead 100) that extends through element 94. Transducer 92 may also again be configured to transmit acoustic energy 90 along a third path (illustrated by arrowhead 102) through element 94 and the received acoustic energy 98 may comprise a portion of the transmitted acoustic energy 102 reflected by tissue 12. The portions 104, 106 of lens 86 on either side of element 94 preferably have similar acoustic impedance and may be identical in composition. Lens 86 may be curved as schematically illustrated to focus the optical energy 88 and/or the acoustic energy 90, 98.

Referring again to FIG. 2, in accordance with the present invention the path 54 taken by the therapeutic energy 52 is aligned with the path 58 taken by the received acoustic energy 56 (and may also be aligned with the path 62 taken by any transmitted acoustic energy 60). In the illustrated embodiment, the paths 54, 58, 62 are aligned in such a way that the paths 54, 58, 62 overlap. In particular, path 54 taken by therapeutic energy 56 includes at least a portion of the paths 58, 62 taken by the received acoustic energy 60 and any transmitted acoustic energy 60. Moreover, the central axis 108 of the beam of therapeutic energy 52 and the beams of received acoustic energy 56 and any transmitted acoustic energy 60 are the same. Alternatively, the path 54 may be parallel to one or both of paths 58, 62. Because the paths 54, 58, 62 taken by the therapeutic energy 52 and the acoustic energy 56, 60 are aligned and have a known relationship to one another, there is no need to employ conventional methods for registration between diagnostic and therapeutic devices. As a result, device 10 does not require additional components used in most registration procedures (e.g., position sensors of fiducial markers) and also reduces the computational demands on ECU 22. Any interference that may result from generation of both the therapeutic and acoustic energies 52, 56, 60 can be addressed using known techniques for filtering signals and/or time interleaved operation. Although relative relationship of the paths 54, 58, 62 of the therapeutic and received and transmitted acoustic energy 52, 56, 60 have been discussed with reference to the embodiment of device 10 shown in FIG. 2, it should be understood that a similar relationship exists for other embodiments of the device described and illustrated herein.

Referring again to FIG. 1, electrodes 44, 46, on shaft 34 of catheter 16 are provided for a variety of diagnostic and therapeutic purposes including, for example, electrophysiological studies, catheter identification and location, pacing, cardiac mapping and ablation. In the illustrated embodiment, electrodes 44, 46 comprise a pair of ring electrodes. It should be understood, however, that the number, orientation and purpose of electrodes 44, 46 may vary.

Patch electrode 18 function as an RF indifferent/dispersive return for the RF ablation signal generated by ablation generator 20. Electrode 18 may also have additional purposes or uses such as the generation of an electromechanical map. Electrode 18 is made from flexible, electrically conductive material and is configured for affixation to body 14 such that electrode 18 is in electrical contact with the patient's skin.

Ablation generator 20 generates, delivers and controls RF energy used by ablation catheter 16. Generator 20 is conventional in the art and may comprise the commercially available unit sold under the model number IBI-1500T RF Cardiac Ablation Generator, available from Irvine Biomedical, Inc. Generator 20 includes an RF ablation signal source 110 configured to generate an ablation signal that is output across a pair of source connectors: a positive polarity connector which may connect to tip electrode 40; and a negative polarity connector which may be electrically connected by conductors or lead wires to patch electrode 18. It should be understood that the term connectors as used herein does not imply a particular type of physical interface mechanism, but is rather broadly contemplated to represent one or more electrical nodes. Source 110 is configured to generate a signal at a predetermined frequency in accordance with one or more user specified parameters (e.g., power, time, etc.) and under the control of various feedback sensing and control circuitry as is know in the art. Source 110 may generate a signal, for example, with a frequency of about 450 kHz or greater. Generator 20 may also monitor various parameters associated with the ablation procedure including impedance, the temperature at the tip of the catheter, ablation energy and the position of the catheter and provide feedback to the clinician regarding these parameters.

ECU 22 is provided to receive a signal generated by transducer 42 responsive to acoustic energy received by transducer 42 and to determine a characteristic of tissue 12 responsive to the signal and/or generate image data relating to tissue 12 responsive to the signal. ECU 2 preferably comprises a programmable microprocessor or microcontroller, but may alternatively comprise an application specific integrated circuit (ASIC). ECU 22 may include a central processing unit (CPU) and an input/output (I/O) interface through which ECU 22 may receive a plurality of input signals including signals from transducer 42 and generate a plurality of output signals including those used to control display device 24. ECU 22 may also include a memory to record information relating to the delivery of therapeutic energy 52 or the receipt and/or transmission of acoustic energy 56, 60. In accordance with one aspect of the present invention, ECU 22 may be programmed with a computer program (i.e., software) encoded on a computer storage medium for determining a degree of coupling between an electrode on a catheter and tissue in a body. The program includes code for determining one or more characteristics of tissue 12 and/or code for generating an image of tissue 12. ECU 22 may form part of a system (not illustrated) for visualization, mapping and navigation of internal body structures such as the system having the model name EnSite NavX™ and commercially available from St. Jude Medical., Inc. and as generally shown with reference to commonly assigned U.S. Pat. No. 7,263,397 titled “Method and Apparatus for Catheter Navigation and Location and Mapping in the Heart,” the entire disclosure of which is incorporated herein by reference. ECU 22 may also form parts of additional systems including, for example, a system (not shown) for use in monitoring and display of electrophysiology data such as an electrogram. ECU 22 may determine a variety of characteristics associated with tissue 12 including, for example, the state of necrosis of tissue 12 resulting from ablation of tissue 12 by therapeutic energy 52 from electrode 40. The determined characteristic of tissue 12 or an image of tissue 12 may be used to assess the effectiveness of the therapeutic procedure and whether the procedure is within predetermined safety thresholds. A clinician, or ECU 22 through a programmed, closed-loop response, may control delivery of therapeutic energy 52 from electrode responsive to this information. Although the functionality of ECU 22 has been described herein with reference to the embodiment of device 10 shown in FIG. 2, it should again be understood that ECU 22 may function similarly with any of the embodiments of device 10 described or illustrated herein.

Display device 24 may be provided to present an image (e.g., a two dimensional or three dimensional image) of tissue 12. Device 24 may also provide a variety of information relating to visualization, mapping and navigation as is known in the art including measures of electrical signals and three-dimensional reconstructions of the tissue 12. Device 24 may comprise an LCD monitor or other conventional display device.

A device in accordance with the present teachings offers one or more of a number of advantages. In particular, the device eliminates the need for registration of conventional diagnostic and therapeutic devices used together in procedures on body tissues by aligning the energy sources and the paths taken by energy produced by the energy sources. In view of the alignment, the relative spatial coordinates of the diagnostic and therapeutic devices are known. By eliminating the need for registration of the two devices, the addition of components used to track position information can be eliminated and the computational demands on the system reduced.

Although several embodiments of this invention have been described above with a certain degree of particularity, those skilled in the art could make numerous alterations to the disclosed embodiments without departing from the scope of this invention. All directional references (e.g., upper, lower, upward, downward, left, right, leftward, rightward, top, bottom, above, below, vertical, horizontal, clockwise and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not as limiting. Changes in detail or structure may be made without departing from the invention as defined in the appended claims.

What is claimed is:

1. A device for both providing therapy to a tissue and detecting a characteristic of said tissue, comprising:
a deformable, tubular body;
an electrode supported by said body and configured to deliver therapeutic energy to said tissue along a first path; and,
an acoustic transducer supported by said body and configured to receive acoustic energy along a second path
wherein said first path and said second path are aligned.

2. The device of claim 1, further comprising an electronic control unit configured to receive a signal generated by said acoustic transducer responsive to said acoustic energy.

3. The device of claim 2 wherein said electronic control unit is further configured to determine said characteristic of said tissue responsive to said signal.

4. The device of claim 3 wherein said characteristic comprises a state of necrosis of said tissue.

5. The device of claim 3 wherein said electronic control unit controls delivery of said therapeutic energy responsive to said characteristic of said tissue.

6. The device of claim 2 wherein said electronic control unit is further configured to generate image data relating to said tissue responsive to said signal.

7. The device of claim 1 wherein said therapy comprises ablation of said tissue.

8. The device of claim 1 wherein said therapeutic energy comprises electromagnetic radiation.

9. The device of claim 8 wherein said electromagnetic radiation comprises radio waves.

10. The device of claim 1 wherein said therapeutic energy comprises acoustic energy.

11. The device of claim 10 wherein said acoustic energy comprises high intensity focused ultrasound (HIFU).

12. The device of claim 10 wherein said electrode and said acoustic transducer have a common piezoelectric layer.

13. The device of claim 12 wherein a portion of said common piezoelectric layer forming a part of said electrode is isolated from a portion of said common piezoelectric layer forming a part of said ultrasonic transducer.

14. The device of claim 1 wherein said therapeutic energy comprises thermal energy.

15. The device of claim 1 wherein said therapeutic energy comprises laser energy.

16. The device of claim 15 wherein a reflective element alters the direction of said first path traveled by said therapeutic energy and said second path extends through said reflective element.

17. The device of claim 1 wherein said therapeutic energy comprises electrical energy sufficient to cause electroporation of a cell membrane in said tissue.

18. The device of claim 1 wherein said second path extends through a wall of said electrode.

19. The device of claim 1 wherein said second path extends through an aperture in said electrode.

20. The device of claim 1 wherein said first and second paths are parallel.

21. The device of claim 1 wherein said first and second paths overlap.

22. The device of claim 1 wherein said first path includes a portion of said second path.

23. The device of claim 1 wherein said acoustic transducer is further configured to transmit acoustic energy toward said tissue along a third path and said received acoustic energy comprises a portion of said transmitted acoustic energy reflected by said tissue.

24. The device of claim 23 wherein said first and third paths are parallel.

25. The device of claim 23 wherein said first and third paths overlap.

26. The device of claim 23 wherein said first path includes a portion of said third path.

27. A device for both delivering therapy to a tissue and detecting a characteristic of said tissue, comprising:
a deformable, tubular body;
an electrode supported by said body and configured to deliver therapeutic energy to said tissue along a first path; and,
an acoustic transducer supported by said body and configured to receive acoustic energy along said first path.

28. A device for both delivering therapy to a tissue and detecting a characteristic of said tissue, comprising:
a deformable, tubular body;
means, supported by said body, for delivering therapeutic energy to said tissue along a first path; and,
means, supported by said body, for receiving acoustic energy along said first path.
